(19) United States
(12) Patent Application Publication
(10) Pub. No.: US 2010/0008976 A1
(43) Pub. Date: Jan. 14, 2010

(54) Medical Use of N-Phenylpropenoyl-Amino Acid Derivatives and Related Compounds

(75) Inventors: Andreas Hensel, Munster (DE); Thomas Hofmann, Munster (DE); Alexandra Deters, Munster (DE); Timo Stark, Munster (DE)

Correspondence Address: OCCHIUTI ROHLICEK & TSAO, LLP, 10 FAWCETT STREET, CAMBRIDGE, MA, 02138, US

(73) Assignee: Westfallsche Wilheims Universitat Munster, Munster (DE)

(21) Appl. No.: 12/373,963

(22) PCT Filed: Jul. 16, 2007
(86) PCT No.: PCT/EP2007/057324

§ 371 Date: Jul. 28, 2009

(30) Foreign Application Priority Data
Jul. 17, 2006 (DE) 102006033321.7

(51) Int. Cl. A61K 9/127; C07C 233/01; A61K 31/165; A61K 31/195; A61K 31/403

(52) U.S. Cl. 424/450; 564/182; 514/617; 514/563; 514/419

(57) ABSTRACT

The invention relates to compounds having the general structural formula (formula I) for use in a diagnostic method or a method for treatment of the human or animal body by surgery or therapy.

The present invention relates to the medical, cosmetic and food-industry use of N-phenylpropenoyl-amino acid derivatives and related compounds. These compounds are secondary plant products which can be isolated in particular from the cacao plant (Theobroma cacao).

The health benefits of secondary plant products have been known for a long time. Antimicrobial or antioxidative properties have been described for a large number of substances, e.g. for polyphenols such as epicatechin, which is obtained from the cacao plant.

Rosmarinic acid is likewise known. This is 1-carboxy-2-(3,4-dihydroxyphenyl)ethyl 3-(3,4-dihydroxyphenyl)acrylate. This compound and derivatives thereof exhibit remarkable properties, e.g. antibacterial, antiviral, antiinflammatory, antigonadotropic and antioxidative properties.

However, under physiological conditions, these compounds are relatively quickly hydrolyzed by endogenous esterases, so that only small amounts enter the bloodstream on administration, and the residence time is only very short. It is therefore possible to reach only low blood titers which are often below the threshold necessary for a possible therapeutic benefit.

A new group of secondary plant products, the N-phenylpropenoyl-amino acid derivatives, was described for the first time in the article [1] by Stark and Hofmann (2005), “Isolation, structure determination, synthesis, and sensory activity of N-phenylpropenoyl-L-amino acids from cocoa (Theobroma cocoa)”, J. Agric. Food Chem; 53: 5419-5428. These compounds consist of an optionally substituted phenylpropenoic acid residue which is connected by an amide linkage to an amino acid residue.

It is an object of the present invention to provide secondary plant products with medical indications which exhibit a longer residence time in the body and have similar or better or additional medical, cosmetic and food-industry properties to or than previously disclosed secondary plant products.

This object is achieved with the features of the present main claim. The dependent claims show preferred embodiments.

Accordingly, the use of compounds of the general structural formula

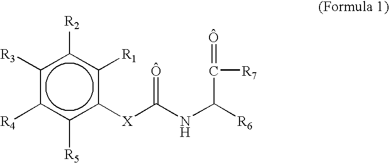

in a surgical, therapeutic or diagnostic method for the treatment of the human or animal body is provided.

Such a medical use of compounds which are covered by this general formula is described for the first time in the present invention. Concerning experimental indications of possible medical indications, reference is made to the examples.

These compounds have great structural similarities with substituted cinnamic esters which frequently occur in nature (rosmarinic acid, chlorogenic acid, etc.) and derivatives thereof. However, in the compounds of the invention the two basic molecules are connected by an amide linkage which is very much more stable under physiological conditions than is the ester linkage in the substituted cinnamic esters which, as already mentioned, are rapidly hydrolyzed by the ubiquitous esterases in the body.

The compound of formula 1 preferably has at the R₁-R₅ positions hydrogen radicals (H—), hydroxy groups (HO—), methoxy groups (CH₃O—) and/or ethoxy groups (C₂H₅O—) and/or bridging methylenedioxy (—O—CH₂—O—) and/or glycosyl groups.

In this connection, the radicals R₃ and/or R₄ are particularly preferably hydroxy groups and/or methoxy groups, while the other radicals are preferably hydrogen radicals.

R₆ is preferably the organic residue of an amino acid, and R₇ is a hydroxy group (—OH) or the N terminus of a peptide.

The term “organic residue of an amino acid” refers hereinafter to the organic residue which is located on the carbon atom carrying the amide group of the amino acid.

If, for instance, the amino acid is alanine, R₆ would be a methyl group; in the case of phenylalanine, R₆ would be a phenyl group, etc.

R₆ is particularly preferably the organic residue of an α-amino acid and/or an L-amino acid.

R₇ may, however, also be a further amino acid which is linked by an amide linkage, or a peptide having a final carboxy terminus.

X is preferably a CₙY₂ₙ radical or a CₙYₙ radical with n=0−6, where Y may be hydrogen radicals (H—) or alkyl groups (e.g. CH₃, C₂H₅, C₃H₇), or a substituted nitrogen, oxygen or sulfur atom. In the case of a CₙYₙ radical with multiple bonds, both the E and the Z configuration can be included.

A precondition for the physiological effects and thus the suitability for any medical indications is very probably on the one hand the amide linkage, which is very much more stable in particular under physiological conditions than, for example, an ester linkage, and therefore in particular makes it possible for the compounds of the invention to be absorbed through the small intestinal epithelium into the bloodstream, and a sufficiently long residence time of the compounds of the invention in the bloodstream.

On the other hand, the presence of the carboxyl group, which is provided by the amino acid, the peptide or the hydroxy group at R₇, appears to be a precondition for the physiological activity.

The chain length of the radical X moreover appears to be equally important, and should not exceed a length of n=0−6.

X is particularly preferably a CₙYₙ radical with n=2, i.e. X is then an HC═CH radical. In this case, the compound of the invention is an N-phenylpropenoyl-amino acid. Such a compound has the following general structural formula:

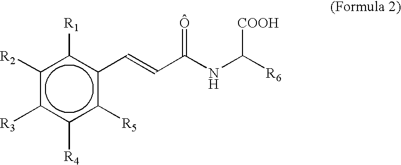

In chemical terms, this is an amide compound composed of an optionally substituted cinnamic acid (phenylpropenoic acid or phenylacrylic acid) and an amino acid. If the phenoyl radical is substituted with a hydroxy group at each of R₂ and R₃, the result is caffeic acid. The amide linkage is formed between the amino group of the amino acid and the carboxyl group of the cinnamic acid or caffeic acid.

R₆ is preferably the organic residue of an amino acid selected from the group including aliphatic, aromatic, polar, basic, acidic, proteinogenic, non-proteinogenic amino acids, α-, β-, γ-, amino acids and/or L- or D-amino acids.

In this connection, the residues of the α-amino acids aspartate, glutamate, tyrosine, tryptophan and dopa are particularly preferred.

In another, likewise preferred embodiment, x=0. Such a compound has the following general structural formula:

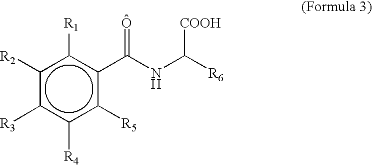

In chemical terms, it is in this case an amide compound composed of an optionally substituted benzoic acid and an amino acid.

The use of a compound as claimed in any of the preceding claims for the manufacture of a medicament for the prevention and/or treatment of bacterial, viral or mycological infections is further provided.

Investigations by the inventors have surprisingly shown that the compounds of the invention display an antiadhesive effect in relation to the colonization by bacteria, viruses and fungi of surfaces such as, for example, the skin, the mucous membranes, the esophagus, the stomach wall and the small intestinal epithelium.

This effect has been shown by way of example with Helicobacter pylori on the gastric mucosa. Concerning this, reference is made to the examples. There are in addition indications of an antiadhesive effect on Campylobacter jejuni, adherent E. coli, Porphyromonas gingivalis, Staphylococcus aureus and Candida albicans. These results suggest that there is a general antiadhesive effect on microorganisms, especially gram-positive and gram-negative bacteria, viruses and fungi.

One cause of this effect appears to be that the compounds of the invention inhibit the formation of a microbial colonization matrix (frequently consisting of polysaccharides and glycoproteins), or that they interact with microbial, in particular bacterial, adesins, or block the receptor functions which are responsible for the adhesion on the epithelial side of the surface to be colonized.

These properties might be of benefit for example for the manufacture of a medicament for the treatment and prophylaxis of chronic gastric mucosal inflammation or for the prevention of infection, e.g. in cases of cutaneous burns, or for poorly healing wounds (e.g. ulcer).

Further potential indications will be directly evident to the skilled worker.

The use of a compound as claimed in any of the preceding claims for the manufacture of a medicament for hepal regeneration, for improving cell metabolism and/or for improving cell proliferation is thus likewise a preferred embodiment of the invention.

In this connection, experiments by the inventors have shown that the compounds of the invention exert a proliferation-promoting effect on human keratinocytes without at the same time influencing the expression of cellular growth factors. Concerning this, reference is made to the examples.

The inventors have shown that the compounds have marked effects on human liver cells and are able to increase markedly the energy production and the cell proliferation. It has further been shown that the compounds of the invention cause an increase in mitochondrial activity of liver cells. Concerning this, reference is made to the examples.

Possible indications in this connection are the regeneration of liver cells which have been damaged by chemotherapeutics, radiation treatment, medicament treatment, toxic effects of medicaments, alcohol abuse, drug abuse, poisonings (especially poisonings by fungi), liver infections (especially hepatitis).

Further possible uses are an increase in the mitochondrial activity, especially of cytochrome P450, which acts as a cellular detoxication enzyme.

Further indications are the use of the compounds of the invention for the manufacture of a medicament for promoting wound healing, skin regeneration, mucosal regeneration, increased proliferation of integumentary appendages (e.g. hair) and cartilage formation, for the prevention and therapy of decubitus ulcer and scarring, or for skin and tissue regeneration, e.g. after (chemical) burns, bedsores etc.

Owing to the antiadhesive effect, use of a compound of the invention for the manufacture of a medicament to counter oral plaque is likewise conceivable.

The invention further provides for the use of a compound as claimed in any of the preceding claims as dietary supplement and functional food.

Of significance in this connection is that, besides the effects mentioned (antiadhesive, cell proliferation-promoting, cell metabolism-increasing), the compounds of the invention have, like many secondary plant products, very probably an antioxidative effect.

Additional factors are that the compounds of the invention are soluble in water and therefore can be admixed in high concentrations also with low-fat, fat-free or calorie-reduced food products, and that they are easy to isolate and synthesize. All these properties make the compounds of the invention appear suitable for use in so-called functional food and as dietary supplement.

The use of a compound of the invention as addition to a cell culture medium is also provided. The mentioned cell proliferation-promoting effect makes the compounds of the invention appear suitable in particular for use in in vitro cell-growing cultures, especially in the production of artificial tissues and organs, for skin models or autologous implant systems. The cell proliferation-promoting effect appears in this connection to relate both to human, animal and plant cell cultures.

A possible approach to an explanation of this is that the compounds of the invention occur naturally especially in plant seeds (such as, for example, cocoa beans), and there promote cell division after germination.

Owing to the abovementioned antiadhesive effect, an additionally preferred use of a compound of the invention is provided for preventing the formation of microbial deposits on surfaces.

This applies in particular to implants, prostheses, catheters (especially pulmonary and bladder catheters), cannulas, surgical and diagnostic instruments (especially endoscopes) and dental prostheses. It would be possible in this way in particular to counter the spread of hospital germs which are frequently resistant to antibiotics.

Further areas of use are sewage pipes, pipelines for food products such as, for example, milk, pipelines in sanitary installations and swimming baths (especially whirlpools), antifouling paints for ships, surfaces of diapers, plasters and other hygiene articles, and cosmetic instruments such as, for example, toothbrushes.

Owing to the high chemical stability, the compounds of the invention are very suitable precisely for these areas of use on exposed surfaces.

The use of a compound of the invention as skincare or oral care agent is also preferably provided. Skin creams, antiaging products, products for preventing scarring or for the treatment of burns and sunburns, dental creams, mouthwashes and the like are intended in this connection. Depending on the use, moreover, the antiadhesive, the cell proliferation-promoting, the cell metabolism-increasing or the antioxidative effect of the compounds of the invention is central.

The invention further provides a medicament, cosmetic composition, skincare composition, composition for the treatment of surfaces, or a dietary supplement or functional food which comprises a compound of the invention.

Also provided is a diagnostic, therapeutic or cosmetic presentation which comprises a compound of the invention, and is in the form of an aqueous extract, of a solution, of an emulsion, of a suspension, of a pulmonary inhalation, of an implant, of a water-oil emulsion, an oil-water emulsion, of a gel, of a tablet, of a capsule, of a cream, of an ointment, of a plaster, of a microemulsion, of a nanoemulsion, as transferosomes or encapsulated in liposomes, micelles or microspheres.

A process is provided for the isolation and processing of a compound of the invention and includes the steps of obtaining plant material, preparing an aqueous or hydroalcoholic extract, centrifuging the extract, where appropriate lyophilizing the extract, and purifying the extract by means of chromatographic processes (e.g. IEC, GPC, adsorption chromatography, partition chromatography) and/or ultrafiltration processes until the concentration is at least 1 g per 100 g of extract.

The plant material to be subjected to extraction is preferably plant material from plants from the list detailed hereinafter. For the aqueous extraction it is moreover possible for example to incubate 1 g of dried plant material with 15 ml of double-distilled water for 2×15 min. The centrifugation can take place for example at 5000×g for 10 min.

It may further be mentioned that the inventors have also developed a synthesis process for preparing the compounds of the invention. Concerning this, reference is made to publication [1].

### DRAWINGS AND EXAMPLES

The present invention is explained in more detail by the examples and figures shown and discussed below. It must be taken into account in this connection that the examples and figures have only a descriptive character and are not intended to restrict the invention in any way.

### Table 1

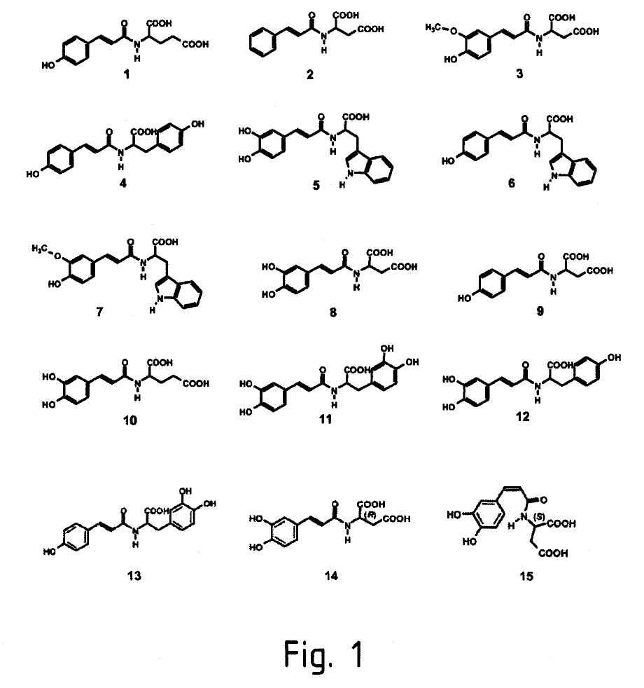

Table 1 shows various compounds of the invention which fall within the scope of protection of the present invention, and which have been isolated from various plants. The structural formulae of the same compounds are shown in FIG. 1. These are all N-phenylpropenoyl-amino acids (see formula 2). Thus, as shown in formula 1 in claim 1, X is, an HC═CH radical, while R₇ is an OH group. The chemical names are evident from Table 1.

TABLE 1

| Substance | No. |
|---|---|
| (−)-N-[4′-hydroxy-(E)-cinnamoyl]-L-glutamic acid | 1 |
| (+)-N-[(E)-cinnamoyl]-L-aspartic acid | 2 |
| (+)-N-[4′-hydroxy-3-methoxy-(E)-cinnamoyl]-L-aspartic acid | 3 |
| (−)-N-[4′-hydroxy-(E)-cinnamoyl]-L-tyrosine | 4 |
| (+)-N-[3′,4′-dihydroxy-(E)-cinnamoyl]-L-tryptophan | 5 |
| (+)-N-[4′-hydroxy-(E)-cinnamoyl]-L-tryptophan | 6 |
| (+)-N-[4′-hydroxy-3-methoxy-(E)-cinnamoyl]-L-tryptophan | 7 |
| (+)-N-[3′,4′-dihydroxy-(E)-cinnamoyl]-L-aspartic acid | 8 |
| (+)-N-[4′-hydroxy-(E)-cinnamoyl]-L-aspartic acid | 9 |
| (−)-N-[3′,4′-dihydroxy-(E)-cinnamoyl]-L-glutamic acid | 10 |
| (−)-N-[3′,4′-dihydroxy-(E)-cinnamoyl]-3-hydroxy-L-tyrosine | 11 |
| (−)-N-[3′,4′-dihydroxy-(E)-cinnamoyl]-L-tyrosine | 12 |
| (−)-N-[4′-hydroxy-(E)-cinnamoyl]-3-hydroxy-L-tyrosine | 13 |
| (−)-N-[3′,4′-dihydroxy-(E)-cinnamoyl]-D-aspartic acid | 14 |
| (+)-N-[3′,4′-dihydroxy-(Z)-cinnamoyl]-L-aspartic acid | 15 |

### Example 1

Dried material from the plants detailed in list 1 was subjected to an aqueous extraction in accordance with the process described above. The extracts obtained were then subjected to an HPLC (high pressure liquid chromatography), an LCMS (liquid chromatography/mass spectrometry) or an NMR (nuclear spin resonance spectrometry). Also measured in this connection as markers were synthesized, deuterium-labeled analogs of compounds 2, 4, 5, 7, 8, 9, 11 and 12 from Table 1.

List 1

Acorus calamus, Angelica archangelica, Arnica montana, Arnica chamissonis, Betula spec.,
Cassia angustifolia, Cassia senna, Cinnamomum ceylanicum, Cola nitida, Coriandrum
sativum, Crocus sativus, Eucalyptus spec., Gentiana lutea, Hedera helix, Hypericum
perforatum, Ilex paraguariensis, Illicum verumm, Juniperus communis, Lavandula spec.,
Matricaria recutita, Pausinystalia yohimbe, Physostigma venenosum, Primula veris, Primula
elatior, Ricinus communis, Salix sp., Sambucus nigra, Silybum marianum, Syzygium
aromaticum, Theobroma cacao, Thymus vulgaris, Thymus zygis, Trigonella foenum-graecum

The compounds of the invention were detectable in the following plants (numbering of the compounds as in Table 1.

TABLE 2

| Plant | Part | (Compound No.) μg/g |
|---|---|---|
| Acorus calamus | rhizome | (2) 0.10 |
| Angelica archangelica | root | (9) 0.73; (1) 0.05; (2) 2.29 |
| Arnica montana, | flower | (3) 0.02; (2) 0.08 |
| Arnica chamissonis | | |
| Cassia angustifolia, | fruit | (3) 1.22 |
| Cassia senna | | |
| Cola nitida | seed | (2) 0.11 |
| Coriandrum sativum | fruit | (8) 1.43; (9) 3.96; (1) 0.03; |
| | | (3) 1.75; (2) 1.24 |
| Hedera helix | leaf | (4) 0.013; (2) 0.16; (5) 0.21; |
| | | (6) 3.49; (7) 0.03 |
| Hypericum perforatum | herb | (1) 0.49 |
| Lavandula spec. | flower | (8) 0.72; (9) 3.67; (1) 0.36; |
| | | (3) 0.54; (2) 4.36 |
| Physostigma venenosum | fruit | (9) 0.82; (1) 0.08; (3) 0.19 |
| Sambucus nigra | flower | (8) 1.29; (9) 3.95; (2) 0.56; |
| | | (1) 1.09; (3) 0.92 (2) 3.42 |
| Silybum marianum | fruit | (5) 0.04 |
| Theobroma cacao | seed | see reference 8 |
| Thymus vulgaris, | herb | (2) 0.09 |
| Thymus zygis | | |

### Examples 2-4

In order to investigate possible pharmacological properties of the compounds of the invention, compounds No. 5 ((+)-N-[3′,4′-dihydroxy-(E)-cinnamoyl]-L-tryptophan; caffeic acid-L-tryptophan as example of an aliphatic amino acid residue at R₆), and No. 8 ((+)-N-[3′,4′-dihydroxy-(E) cinnamoyl]-L-aspartic acid; caffeic acid-L-aspartate as example of an aromatic amino acid residue at R₆) were used.

### Example 2

### Investigations on a Human Liver Cell Line

The HepG2 cell line, clone H20, was received from Prof. Mersch-Sundermann, Giessen University, and cultured as described in [2]. The cells were cultured in low glucose (1 g/l) Dulbecco's modified Eagle's medium (DMEM) with L-glutamine and 25 mM Hepes, which was mixed with 15% (v/v) heat-inactivated fetal calf serum (FCS) and gentamycin (30 μg/ml), in a moist atmosphere at 37+/−0.5° C., 5% CO₂. The cells were trypsinized, washed with PBS (pH 7.4), gently centrifuged and then a cell suspension in a cell medium was prepared by forcing the sediment through a needle. The medium was changed every three to five days. The compounds Nos. 5 and 8 to be tested were dissolved in a concentration of 1 mg/ml in HepG2 medium, to which the serum-free supplement SerEx was added instead of FCS, and filtered through a 0.2 μm cellulose acetate filter.

The cells were seeded in 96-well microtiter plates (1×10⁴ cells/well). After 24 h, the medium was removed and the cells were exposed to the compounds to be tested, in concentrations of 100 and 10 μg/ml, for 48 h. The cell metabolic activity was quantified as 2.5 mg/ml MTT in accordance with [3]. The extracytosolic LDH [4] was quantified with a cytotoxicity assay.

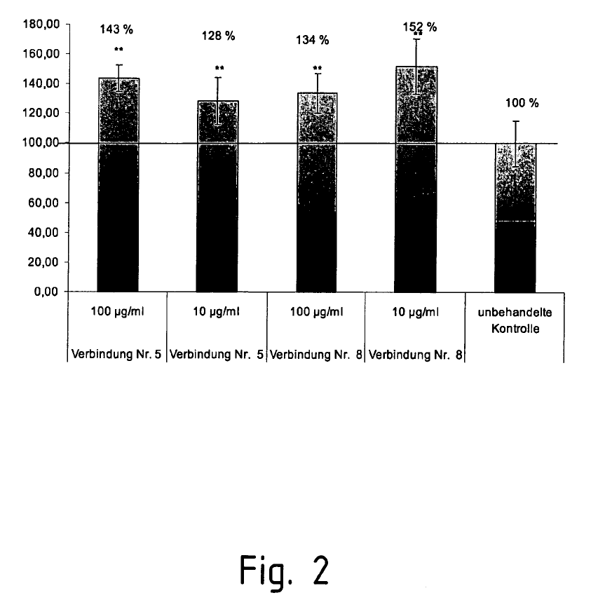

Both compound No. 5 and compound No. 8 increased the mitochondrial activity after incubation for 48 hours (see FIG. 2). A shortened incubation time of 24 hours showed that compound No. 8 significantly increased the energy status, whereas compound No. 5 showed no effect. It was possible to establish that compound No. 8 shows a rapid and vigorous stimulating effect. Determination of LDH showed that no necrotic cytotoxicity was detectable.

### Example 3

### Investigations on a Human Keratinocyte Cell Line

Human primary keratinocytes (NHK) were isolated from human skin obtained by surgical resection of Caucasian patients. In vitro tests relating to the mitochondrial activity [3], the BrdU incorporation [5] and for necrotic effects by means of an LDH assay [4] were carried out.

For a quantitative real-time PCR, the NHK were incubated with the compounds to be tested, which were dissolved in serum-free keratinocyte medium, for 6 hours. Keratinocyte medium with various growth factors was used as positive control. The total RNA was purified using the Perfect RNA eukaryotic mini kit. RNA aliquots were prepared for reverse transcription PCR (RT-PCR), which was carried out using TaqMan Reverse Transcription Reagents®.

The quantitative RT-PCR was carried out using the TaqMan Universal PCR Master Mix and specific TaqMan gene expression assays for KGF, the KGF receptor, the EGF receptor, the insulin receptor, STAT6 and 18srRNA as endogenous control in a 7300 Real-Time PCR System from Applied Biosystems.

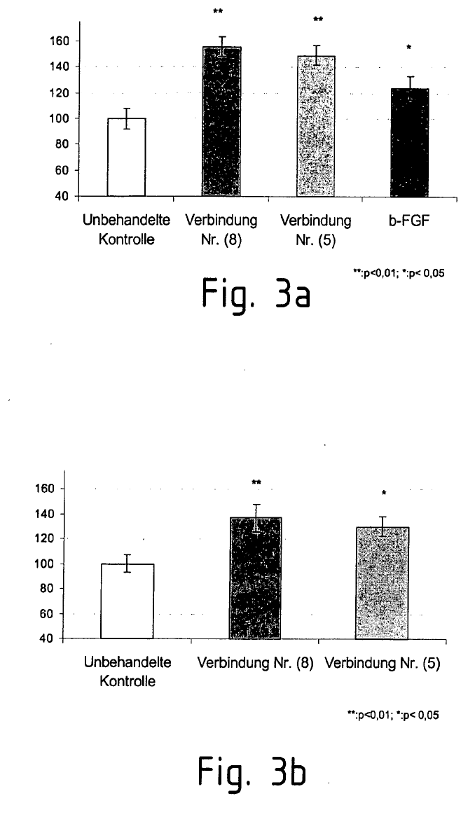

The investigations were carried out with cells of the 2nd to 6th passage. Both compound No. 5 and compound No. 8 increased in a concentration of 10 μg/ml both the mitochondrial activity and the proliferation (FIG. 3). No cytotoxicity was detected.

Since in many cases effects on the physiology of keratinocytes is mediated by an increased expression of growth factors or growth factor receptors, the influence of compounds Nos. 5 and 8 on the expression of the gene of the keratinocyte growth factor KGF, its receptor (KGFR), the epidermal growth factor receptor EGFR and the insulin receptor InsR was investigated by quantitative RT-PCR. Expression of the transcription factor STAT6 and of the gene for involucrin (a specific protein for early cell differentiation) was also investigated.

No identifiable effect on the expression of KGF, KGFR, EGFR, InsR and involucrin was observed. A significantly increased expression was detected for STAT 6 (9× higher by comparison with the controls).

### Example 4

### Investigations on the Adhesion of Helicobacter pylori

Adhesion tests were carried out in accordance with [6,7]. This entailed FITC-labeled bacteria being incubated with the compounds to be tested (1 mg/ml). Deparaffinized pieces of stomach tissue were incubated with the bacteria. Microorganisms which adhere to the epithelium were counted under a fluorescence microscope and compared with an untreated control.

The maximum adhesion, as found for example in the untreated control groups (negative control), was assigned a score of +++++, while lower adhesions were assigned scores ++++, +++, ++, + or −, the score found for the positive control (sialyllactose, [7]) being −.

A strong and reproducible antiadhesive effect on Helicobacter pylori with almost complete suppression of adhesion was observed in particular after incubation of the bacteria with compound No. 8 (1 mg/ml), while compound No. 5 showed no effect. In order to investigate a direct cytotoxicity of the compounds to be tested on H. pylori, the compounds were tested in concentrations of 2.5 mg/ml on the microorganism in a disk diffusion assay (positive control with 0.5 μg amoxicillin). No signs of a bacteriocidal or bacteriostatic effect of the compounds to be tested were found in this case.

The experiments described in examples 2-4 were also carried out with compound No. 11. However, this showed none of the effects described. Nevertheless, the results shown with compounds Nos. 5 and 8, which were selected because of their exemplary structures, indicate that other compounds of the invention of formula 1 or 2, or FIG. 1, will show similar effects. It is therefore permissible to claim the medical indications mentioned for all the compounds falling under the formulae mentioned.

### REFERENCES

(1) Stark T, Hofman T. Isolation, structure determination, synthesis, and sensory activity of N-phenylpropenoyl-L-amino acids from cocoa (Theobroma cocoa). J. Agric. Food Chem. 2005; 53; 5419-5428
(2) Dauer A, Hensel A, Lhoste F, Knasmueller S, Mersch-Sundermann V. Genotoxic and antigenotoxic effects of catechin and tannins from the bark of Hamamelis virginiana L. in metabolically competent, human hepatoma cells (HepG2) using single cell electrophoresis. Phytochem. 2003; 63: 199-207
(3) Mosmann M. Rapid calorimetric assay for cellular growth and survival: applications to proliferation and cytotoxicity assays. J. Immun. Meth. 1983; 65: 55-63
(4) Martin A, Clynes M. Comparison of 5 microplate calorimetric assays for in vitro cytotoxicity testing and cell proliferation assays. Cytotechnol. 1993; 11: 49-58
(5) Porstmann T, Ternyk T, Avrameas S. Quantification of 5-bromo-2′-deoxyuridine into DNA: an enzyme immunoassay for the assessment of the lymphoid cell proliferative response. J. Immun. Meth. 1985: 82:
(6) Lengsfeld C, Deters A, Faller G, Hensel A. High molecular weight polysaccharides from black currant seeds inhibit adhesion of Helicobacter pylori to human gastric mucosa. Planta Med. 2004; 70: 620-626
(7) Lengsfeld C, Titgemeyer F, Faller G, Hensel A. Glycosylated compounds from okra inhibit adhesion of Helicobacter pylori to human gastric mucosa. J. Agric. Food Chem. 2004; 52: 1495-1503
(8) Stark T, Justus H, Hofmann T. A stable isotope dilution analysis (SIDA) for the quantitative determination of N-phenylpropenoyl-L-amino acids in coffee beverage and cocoa. J. Agric. Food Chem. 2006; 54: 2859-2867.

### DRAWINGS

FIG. 1: Structural formulae of some N-phenylpropenoyl-amino acids of the invention

FIG. 2: Influence of compounds No. 5 and No. 8 (10 and 100 μg/ml) on the mitochondrial activity of human liver cells (HEPG2) after incubation for 48 hours.

The measurement plotted in FIG. 2 is the relative mitochondrial dehydrogenase activity (untreated control=100%). The bars represent the means of a representative experiment with n=10.

FIG. 3: Influence of compounds No. 5 and No. 8 (10 μg/ml) on the mitochondrial activity (A) and the mitotic proliferation (B) of HaCaT keratinocytes after incubation for 60 hours.

The measurement plotted in FIG. 3A is the relative mitochondrial dehydrogenase activity (untreated control=100%) and in FIG. 3 b is the relative mitochondrial proliferation rate (untreated control=100%).

The mitochondrial activity was investigated using the MTT assay, and the proliferation by BrdU-incorporation ELISA. The bars represent the standard errors with n=10. Negative control: untreated cells, positive control: fibroblast growth factor FGF.

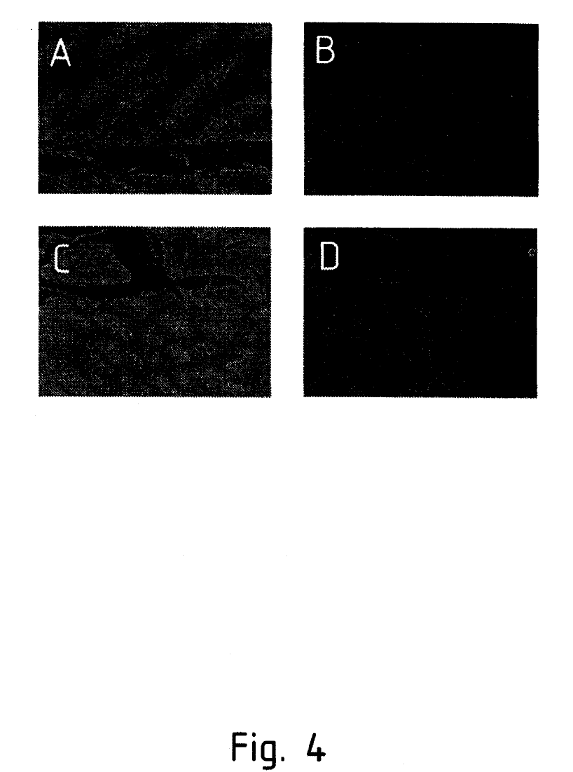

FIG. 4: Fluorescence microscopes (200×) of a representative in situ experiment with FITC-labeled H. pylorion human gastric mucosa: (A) complete adhesion (+++++) of untreated bacteria, fluorescence intensity standardized to 100% (negative control), (B) positive control (−), fluorescence intensity 10% (C) compound No. 5 (++++), fluorescence intensity 78%, (D) compound No. 8 (+) fluorescence intensity 19%.

1-14. (canceled)

15. Compounds of the general structural formula 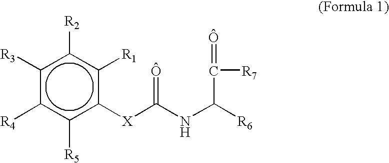
where X is an HC═CH radical, and where the compound has only one phenylpropenoyl group or only one derivative thereof per compound,
for use in a surgical, therapeutic or diagnostic method for the treatment of the human or animal body.

16. Compounds as claimed in claim 15, characterized in that
a) R₁-R₅ are hydrogen radicals (H—), hydroxy groups (HO—), methoxy groups (CH₃O—), ethoxy groups (C₂H₅O—) and/or bridging methylenedioxy (—O—CH₂—O—) and/or glycosyl groups,
b) R₆ is the organic residue of an amino acid,
c) R₇ is a hydroxy group (—OH) or an amino acid which is linked by an amide linkage, or a peptide having a final carboxy terminus.

17. Compounds as claimed in claim 15, characterized in that R₆ is the organic residue of an amino acid selected from the group including aliphatic, aromatic, polar, basic, acidic, proteinogenic, non-proteinogenic amino acids, α-, β-, γ-, amino acids and/or L- or D-amino acids.

18. The use of a compound as in claim 15 for the manufacture of a medicament for the prevention and/or treatment of bacterial, viral or mycological infections.

19. The use of a compound as claimed in claim 15 for regeneration, for improving cell metabolism and/or for improving cell proliferation.

20. The use of a compound as claimed in claim 15 for the manufacture of a medicament to counter oral plaque.

21. The use of a compound as claimed in claim 15 as dietary supplement.

22. The use of a compound as claimed in claim 15 as addition to a cell culture medium.

23. The use of a compound as claimed in claim 15 for preventing the formation of microbial deposits on surfaces.

24. The use of a compound as claimed in claim 15 as skincare or oral care agent.

25. A medicament, cosmetic composition, skincare composition, composition for the treatment of surfaces, dietary supplement, or functional food, comprising a compound as claimed in claim 15.

26. A diagnostic or therapeutic presentation comprising a compound as claimed in claim 15, characterized in that it is in the form of an aqueous extract, of a solution, of an emulsion, of a suspension, of a pulmonary inhalation, of an implant, of a water-oil emulsion, an oil-water emulsion, of a gel, of a tablet, of a capsule, of a cream, of an ointment, of a plaster, of a microemulsion, of a nanoemulsion or encapsulated in liposomes, micelles or microspheres.

27. A process for the isolation and processing of a compound as claimed in claim 15, including the following steps:
a) obtaining plant material,
b) preparing an aqueous or hydroalcoholic extract,
c) centrifuging the extract,
d) where appropriate lyophilizing the extract, and
e) purifying the extract by means of chromatographic processes and/or ultrafiltration processes until the concentration is at least 1 g of a compound as claimed in any of the preceding claims per 100 g of extract.
